(19) United States
(12) Patent Application Publication
(10) Pub. No.: US 2010/0076197 A1
(43) Pub. Date: Mar. 25, 2010

(54) PROCESS FOR RIMONABANT

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Kesireddy Subash Chander Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 11/993,544

(22) PCT Filed: Sep. 11, 2006
(86) PCT No.: PCT/IN06/00345

§ 371 Date: Dec. 21, 2007

(51) Int. Cl. C07D 401/12

(52) U.S. Cl. 546/211

(57) ABSTRACT

The present invention provides an improved and commercially viable process for the preparation of rimonabant substantially free of amide impurity, namely 5-(4-chlorophenyl)-1-(2,4-dichlorophenyl-4-methyl-pyrazole-3-carboxamide and its pharmaceutically acceptable acid addition salts thereof. Thus, for example, 5-(4-chlorophenyl)-1-(2,4-dichlorophenyl-4-methyl-pyrazole-3-carboxylic acid chloride is reacted with 1-aminopiperidine in the presence of a base and optionally a phase transfer catalyst is used such as tetra-butylammonium bromide in a biphasic reaction medium containing water and a water-immiscible solvent to obtain pure rimonabant.

### FIELD OF THE INVENTION

The present invention provides an improved and commercially viable process for the preparation of rimonabant substantially free of amide impurity, namely 5-(4-chlorophenyl)-1-(2,4-dichlorophenyl-4-methyl-pyrazole-3-carboxamide and its pharmaceutically acceptable acid addition salts thereof.

### BACKGROUND OF THE INVENTION

U.S. Pat. Nos. 5,624,941 and 5,462,960 disclosed pyrazole-3-carboxamide derivatives, processes for their preparation, pharmaceutical compositions in which they are present and uses thereof. These compounds possess a very good affinity to the cannabinoid receptor and are useful in the therapeutic areas in which cannabis is known to be involved. The therapeutic indications of cannabinoids concern a variety of areas such as the immune system, the central nervous system and the cardiovascular or endocrine system. Among them, rimonabant, chemically 5-(4-chlorophenyl)-1-(2,4-dichlorophenyl)-4-methyl-N-(piperidin-1-yl)-pyrazole-3-carboxamide is a promising CBₗ receptor antagonist with potent and selective activity in binding and functional assays, and which has been shown to inhibit motivational and consummatory aspects of feeding and reduce alcohol and nicotine intake in animal models. Rimonabant is represented by the following structure:

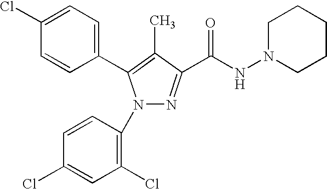

Processes for the preparation of rimonabant and related compounds were disclosed in U.S. Pat. No. 5,624,941 and PCT Publication No. 2006/021652 A1.

According to the U.S. Pat. No. 5,624,941, rimonabant can be prepared, either by (i) reacting 5-(4-chlorophenyl)-1-(2,4-dichlorophenyl-4-methyl-pyrazole-3-carboxylic acid chloride, obtained by reaction of thionyl chloride with 5-(4-chlorophenyl)-1-(2,4-dichlorophenyl-4-methyl-pyrazole-3-carboxylic acid, with 1-aminopiperidine in a solvent such as dichloromethane in an inert atmosphere, at a temperature between 0° C. and room temperature, in the presence of a base such as triethylamine; or (ii) reacting the mixed anhydride of 5-(4-chlorophenyl)-1-(2,4-dichlorophenyl-4-methyl-pyrazole-3-carboxylic acid, obtained by reaction of ethylchloroformate with the said acid in the presence of a base such as triethylamine, with 1-aminopiperidine in a solvent such as dichloromethane in an inert atmosphere, at a temperature between 0° C. and room temperature, in the presence of a base such as triethylamine.

The yields of rimonabant obtained according to the processes described in the U.S. Pat. No. 5,624,941 are very poor and the processes involve column chromatographic purifications. Methods involving column chromatographic purifications cannot be used for large-scale operations, thereby making the process commercially not viable.

According to the PCT Publication No. 2006/021652 A1, rimonabant is prepared by reacting 5-(4-chlorophenyl)-1-(2,4-dichlorophenyl)-4-methyl-pyrazole-3-carbohydrazide, obtained by reaction of 5-(4-chlorophenyl)-1-(2,4-dichlorophenyl-4-methyl-pyrazole-3-carboxylic acid chloride with hydrazine hydrate, with 1,5-dihalogenopentane compound in presence of an organic base, such as a tertiary amine, for example, triethylamine, or of a mineral base, such as NaOH, KOH, K₂CO₃, Na₂CO₃, CS₂CO₃, in a solvent selected from the group consisting of an aromatic solvent, an ether solvent, dioxane and a nitrile solvent.

The preparation of rimonabant as described in the PCT Publication No. 2006/021652 A1 involves a lengthy process, the reaction requires longer time about 45 hours to complete and the yield obtained is not satisfactory. The process described in this publication also involves column chromatographic purifications. Methods involving column chromatographic purifications cannot be used for large-scale operations, thereby making the process commercially not viable.

The U.S. patent appl. No. 2005/0043356 A1 teaches a crystalline polymorph of rimonabant, its method of preparation and the pharmaceutical compositions containing this polymorph.

Rimonabant obtained by the processes described in the art is not satisfactory from a purity point of view. The amide impurity, namely 5-(4-chlorophenyl)-1-(2,4-dichlorophenyl-4-methyl-pyrazole-3-carboxamide is a main concern and rimonabant obtained by the prior art is contaminated with this impurity.

However, a need still remains for an improved and commercially viable process of preparing pure rimonabant that solves the aforesaid problems associated with processes described in the prior art, which will be suitable for large-scale preparation, in terms of simplicity, chemical yield and purity of the product.

Extensive experimentation is carried out by the present inventors to find a way to eliminate this amide impurity. As a result, it has been found that when 5-(4-chlorophenyl)-1-(2,4-dichlorophenyl-4-methyl-pyrazole-3-carboxylic acid chloride is reacted with 1-aminopiperidine in a biphasic reaction medium containing water and a water-immiscible solvent in the presence of a water-soluble base and optionally using a phase transfer catalyst, rimonabant is obtained in high purity and in high yield.

According to the novel process, no chromatographic separations are required for isolating pure rimonabant thereby making the process commercially viable.

### DETAILED DESCRIPTION OF THE INVENTION

In accordance with the present invention, there is provided a process for preparing rimonabant of the formula I:

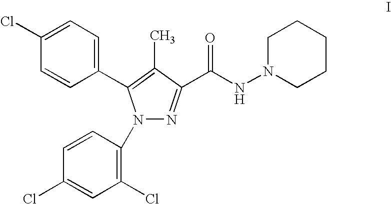

substantially free of the amide impurity, namely 5-(4-chlorophenyl)-1-(2,4-dichlorophenyl-4-methyl-pyrazole-3-carboxamide or a pharmaceutically acceptable salt thereof;
which comprises reacting the acid chloride compound of the formula II:

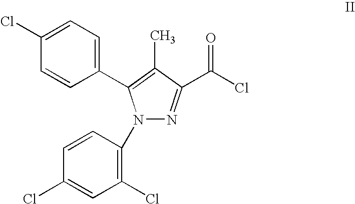

with 1-aminopiperidine in a biphasic reaction medium comprising water and a water-immiscible solvent in the presence of a water-soluble base and optionally using a phase transfer catalyst to give pure rimonabant of the formula I substantially free of the amide impurity and optionally converting rimonabant formed into a pharmaceutically acceptable acid addition salt of rimonabant.

The term “rimonabant substantially free of amide impurity” refers to rimonabant containing a content of amide impurity of less than about 0.1% by weight, preferably less than about 0.05% by weight and still more preferably containing no amide impurity.

The reaction may be carried out between 0° C. and reflux temperature of the solvent used, preferably carried out at about 0-35° C., more preferably at about 10-30° C. and still more preferably at about 10-25° C.

The phase transfer catalyst used in the reaction is selected from the group consisting of ammonium salts such as tetra-n-butylammonium bromide, tetra-n-butylammonium chloride, tetra-n-butylammonium hydroxide, tetra-n-butylammonium iodide, tetraethylammonium chloride, tricaprylylmethyl ammonium chloride, benzyltributylammonium bromide, benzyltriethylammonium bromide, tetramethylammonium chloride, cetyltrimethylammonium bromide, cetylpyridinium bromide, N-benzylquininium chloride, hexadecyltrimethyl ammonium chloride, and octyltrimethylammonium chloride. Preferable phase transfer catalysts used in the reaction are selected from the group consisting of tetra-n-butylammonium bromide, tetra-n-butylammonium chloride, tetra-n-butylammonium hydroxide, tetra-n-butylammonium iodide, and a more preferable phase transfer catalyst is tetra-n-butylammonium bromide.

The phase transfer catalyst may be used in a stochiometric or substochiometric amount, preferably from about 0.03 to about 0.25 mol equivalents with respect to the acid chloride compound of formula II.

The base used in the reaction may be an inorganic base selected from the group consisting of sodium hydroxide, potassium hydroxide, sodium carbonate, sodium bicarbonate, potassium carbonate, potassium bicarbonate, and combinations thereof. A more preferable base is sodium hydroxide. Preferably a solution of inorganic base in water may be used in the reaction.

Preferably, at least 1 molar equivalent of base per 1 mole of acid chloride compound of formula II is used, and more preferably between 5 and 15 molar equivalents of base per 1 mole of acid chloride compound of formula II is used.

The water-immiscible solvent used in the reaction is selected from the group consisting of chlorinated hydrocarbon solvents such as methylene chloride, ethylene dichloride and chloroform; hydrocarbon solvents such as toluene, benzene, n-hexane, n-heptane, xylene and cyclohexane; ester solvents such as ethyl acetate, methyl acetate and isobutyl acetate; and ether solvents such as dimethyl ether, diethyl ether and diisopropyl ether. More preferable solvent is selected from the group consisting of methylene chloride, toluene, ethyl acetate and diisopropyl ether.

The reaction is normally completed within 1 hour 30 minutes and usually within 30 minutes.

After the reaction is completed, the reaction mass may then be subjected to acid base treatment followed by usual work up such as washings, extractions etc.

The novel process provides rimonabant in high yield and purity, thus obviating the need to use column chromatography to purify the material.

Preferable pharmaceutically acceptable acid addition salts of the formula I, are but are not limited to, the salts of rimonabant obtained from hydrochloric acid, hydrobromic acid, hydroiodic acid, methanesulfonic acid and benzenesulfonic acid, and a more preferable salt being rimonabant hydrochloride.

The following examples are given for the purpose of illustrating the present invention and should not be considered as a limitation on the scope or spirit of the invention.

### REFERENCE EXAMPLE

### Step-a

1.5M Lithium hexamethyl disilazane in tetrahydrofuran (2000 ml) is stirred with cyclohexane (2400 ml) at 25-30° C. under N₂ atmosphere, the mass cooled to 15° C. and then a solution of 4-chloropropiophenone (400 gm) in cyclohexane (960 ml) is added for 30 minutes at 15-20° C. The reaction mass is stirred for 5 hours at 15-20° C. under N₂ atmosphere, diethyl oxalate (364 ml) is added to the mass for 20 minutes at 25-30° C. and then stirred for 14 hours at 25-30° C. The solid is filtered, washed with 1000 ml of cyclohexane and then dried to give 400 gm of lithium salt of ethyl 4-(4-chlorophenyl)-3-methyl-4-oxydo-2-oxobuten-3-oate.

### Step-b

The solid obtained in step-a is added to ethanol (2500 ml) at 25-30° C., cooled the mass to 10° C. and then 2,4-dichlorophenylhydrazine hydrochloride is added at 10-15° C. The reaction mass is stirred for 2 hours at 10-15° C., the solid is filtered, washed with ethanol (300 ml) and then the material is dried to give 475 gm of (Z)-ethyl 2-[2-(2,4-dichlorophenyl)hydrazono]-4-(4-chlorophenyl)-3-methyl-4-oxobutanoate.

### Step-c

The solid obtained in step-b and toluene (3206 ml) are added to p-toluenesulfonic acid (41.5 gm) under stirring at 25-30° C., the contents are refluxed for 6 hours 30 minutes at 110-112° C. under azeotropic conditions (collected volume of water: 25 ml) and then cooled to 85° C. To the reaction mass is added carbon (12 gm), cooled to 30° C., filtered on a celite bed and then the bed washed with toluene (1300 ml). The resulting filtrate is washed with 350 ml of saturated sodium chloride solution to obtain ethyl 5-(4-chlorophenyl)-1-(2,4-dichlorophenyl)-4-methyl-pyrazole-3-carboxylate (organic layer volume: 4.75 Lt).

### Step-d

Benzyl trimethylammonium chloride (25 gm) is added to the solution of NaOH (147.2 gm) in water (250 ml) under stirring at 25-30° C. and then the organic layer that resulted from step-c is added at 25-30° C. (pH should be between: 9-10). The reaction mass is heated to 70° C., stirred for 3 hours 30 minutes at 68-70° C. and then cooled to 10° C. To the reaction mass is added the solution of conc. HCl (475 ml) in water (1583 ml) for 30 minutes at 10-15° C. (pH=1.0) and stirred for 1 hour at 10-15° C. The solid is filtered, washed with 300 ml of toluene followed by 500 ml of water and then dried to give 380 gm of 5-(4-chlorophenyl)-1-(2,4-dichlorophenyl-4-methyl-pyrazole-3-carboxylic acid.

### Example

### Step-I

Toluene (700 ml) and dimethylformamide (4 ml) are added to 5-(4-chlorophenyl)-1-(2,4-dichlorophenyl-4-methyl-pyrazole-3-carboxylic acid (100 gm, obtained in step-d of reference example) under stirring at 25-30° C., the solution of thionyl chloride (33 ml) in toluene (100 ml) is slowly added for 30-45 minutes at 25-30° C. and then the contents are stirred for 3 hours at 80-85° C. The reaction mass is distilled under vacuum at below 70° C., to the residue is added toluene (200 ml) and again distilled to give 5-(4-chlorophenyl)-1-(2,4-dichlorophenyl-4-methyl-pyrazole-3-carboxylic acid chloride. The residue is cooled to 30° C. and dissolved in methylene chloride (700 ml) and kept aside.

### Step-II

1-Aminopiperidine (33 gm), 30% NaOH solution (333 ml) and tetra-n-butylammonium bromide (5 gm) are added to methylene chloride (1115 ml) under stirring at 25-30° C., the contents are cooled to 10° C. and then the solution that resulted in the step-I is slowly added for 1 hour to 1 hour 30 minutes at 10-15° C. The reaction mass temperature is raised to 25° C., stirred for 1-2 hours and the layers separated. The resulting organic layer is washed with 10% NaCl solution (300 ml) and then subjected to carbon treatment at 25-30° C. The celite bed is washed with methylene chloride (200 ml), the resulting organic layer is dried on Na₂SO₄, distilled under vacuum at 40° C. and then co-distilled with acetone (100 ml). The residue is dissolved in acetone (725 ml), pH of the mass is adjusted to 2.0 with conc. HCl (25 ml) and then stirred for 30 minutes at 20-25° C. The solid is filtered, washed with 50 ml of acetone and then dried to give 120 gm of 5-(4-chlorophenyl)-1-(2,4-dichlorophenyl)-4-methyl-N-(piperidin-1-yl)-pyrazole-3-carboxamide hydrochloride (rimonabant hydrochloride, HPLC purity: 99.7%).

### Step-III

Rimonabant hydrochloride salt (120 gm, obtained in step-II) and water (300 ml) are added to methylene chloride under stirring at 25-30° C., pH of the mass is adjusted to 10.0 with 1:1 NH₃: H₂O (60 ml) solution at 20-25° C. and then stirred for 10 minutes at 20-25° C. The layers are separated, the organic layer is subjected to carbon treatment at 25-30° C., filtered on a celite bed and then the bed washed with methylene chloride (100 ml). The organic layer is dried on Na₂SO₄, distilled under vacuum at 40° C. and then co-distilled with cyclohexane (100 ml). To the residue is added cyclohexane (480 ml) and stirred for 1 hour at 25-30° C. The solid is filtered, washed with 50 ml of cyclohexane and then dried to give 105 gm of rimonabant (HPLC purity: 99.9%, Content of amide impurity: Not detected).

We claim:

1. A process for the preparation of rimonabant of the formula I: 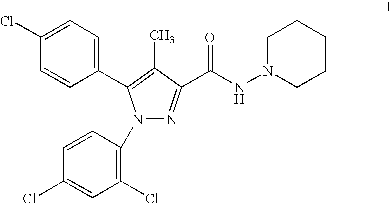
substantially free of amide impurity, namely 5-(4-chlorophenyl)-1-(2,4-dichlorophenyl-4-methyl-pyrazole-3-carboxamide or a pharmaceutically acceptable salt thereof;
which comprises reacting the acid chloride compound of the formula II: 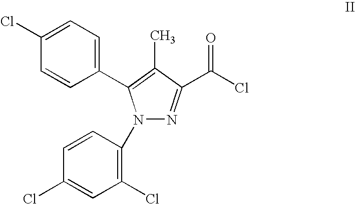
with 1-aminopiperidine in a biphasic reaction medium comprising water and a water-immiscible solvent in the presence of a water-soluble base and optionally using a phase transfer catalyst to give pure rimonabant of the formula I substantially free of amide impurity and optionally converting the rimonabant formed into a pharmaceutically acceptable acid addition salts of rimonabant.

2. The process as claimed in claim 1, wherein the rimonabant obtained has a content of amide impurity of less than about 0.1% by weight.

3. The process as claimed in claim 2, wherein the rimonabant has a amide impurity of less than about 0.05% by weight.

4. The process as claimed in claim 3, wherein the rimonabant contains no amide impurity.

5. The process as claimed in claim 1, wherein the reaction is carried out between about 0° C. and reflux temperature of the solvent used.

6. The process as claimed in claim 5, wherein the reaction is carried out at about 0-35° C.

7. The process as claimed in claim 6, wherein the reaction is carried out at about 10-30° C.

8. The process as claimed in claim 7, wherein the reaction is carried out at about 10-25° C.

9. The process as claimed in claim 1, wherein the phase transfer catalyst is selected from the group consisting of ammonium salts such as tetra-n-butylammonium bromide, tetra-n-butylammonium chloride, tetra-n-butylammonium hydroxide, tetra-n-butylammonium iodide, tetraethylammonium chloride, tricaprylylmethylammonium chloride, benzyltributylammonium bromide, benzyltriethylammonium bromide, tetramethylammonium chloride, cetyltrimethylammonium bromide, cetylpyridinium bromide, N-benzylquininium chloride, hexadecyltrimethyl ammonium chloride, and octyltrimethylammonium chloride.

10. The process as claimed in claim 9, wherein the phase transfer catalyst is selected from tetra-n-butylammonium bromide, tetra-n-butylammonium chloride, tetra-n-butylammonium hydroxide and tetra-n-butylammonium iodide.

11. The process as claimed in claim 10, wherein the phase transfer catalyst is tetra-n-butylammonium bromide.

12. The process as claimed in claim 1, wherein the base is an inorganic base selected from the group consisting of sodium hydroxide, potassium hydroxide, sodium carbonate, sodium bicarbonate, potassium carbonate, potassium bicarbonate, and combinations thereof.

13. The process as claimed in claim 12, wherein the base is sodium hydroxide.

14. The process as claimed in claim 1, wherein the solution of inorganic base in water is used.

15. The process as claimed in claim 1, wherein the water-immiscible solvent is selected from the group consisting of chlorinated hydrocarbon solvents such as methylene chloride, ethylene dichloride and chloroform; hydrocarbon solvents such as toluene, benzene, n-hexane, n-heptane, xylene and cyclohexane; ester solvents such as ethyl acetate, methyl acetate and isobutyl acetate; and ether solvents such as dimethyl ether, diethyl ether and diisopropyl ether.

16. The process as claimed in claim 15, wherein the solvent is selected from the group consisting of methylene chloride, toluene, ethyl acetate and diisopropyl ether.
